(19) United States
(12) Patent Application Publication
(10) Pub. No.: US 2009/0099346 A1
(43) Pub. Date: Apr. 16, 2009

(54) THERMALLY STABLE CRYSTALLINE EPIRUBICIN HYDROCHLORIDE

(76) Inventors: Victor Matvienko, Donetsk (UA); Alexey Matvyeyev, Donetsk (UA); Aleksandr M. Itkin, San Diego, CA (US); Alexander F. Zabudkin, Donetsk (UA)

Correspondence Address: Vista IP Law Group LLP, 2040 MAIN STREET, 9TH FLOOR, IRVINE, CA, 92614, US

(21) Appl. No.: 12/245,755

(22) Filed: Oct. 5, 2008

Related U.S. Application Data
(63) Continuation-in-part of application No. 10/877,221, filed on Jun. 25, 2004, now Pat. No. 7,485,707.
(60) Provisional application No. 60/484,132, filed on Jul. 2, 2003.

(51) Int. Cl. C07H 15/24

(52) U.S. Cl. 536/6.4

(57) ABSTRACT

A crystalline form of epirubicin hydrochloride, named herein as “type II” crystalline epirubicin hydrochloride, has excellent thermal stability. Type II crystalline epirubicin hydrochloride has a powder X-ray diffraction pattern having average values of diffraction angle (2θ) and relative intensity P(%) as presented in the following table:

| Diffraction Angle | Relative Intensity |
|---|---|
| 2Θ | P % |
| 5.2 | 100 |
| 9.2 | 24.5 |
| 10.3 | 14.3 |
| 13.7 | 32.8 |
| 14.6 | 10.9 |
| 15.5 | 49.3 |
| 18 | 19.1 |
| 19.2 | 21.1 |
| 19.4 | 11.3 |
| 20.7 | 19.9 |
| 21.1 | 29.7 |
| 21.4 | 18 |
| 22 | 23.1 |
| 22.5 | 69.6 |
| 23.6 | 20.5 |
| 24.1 | 72.1 |
| 25.8 | 67 |
| 26.1 | 15.9 |
| 27.7 | 47.9 |
| 29.8 | 38 |
| 31.9 | 14.1 |
| 32.1 | 16.7 |
| 36.4 | 16.6 |
| 37.7 | 18.8 |
| 41.1 | 14.5 |

### RELATED APPLICATIONS

This is a continuation-in-part of U.S. patent application Ser. No. 10/877,221, filed on Jun. 25, 2004, now U.S. patent Ser. No. ______, which claims the benefit of U.S. Provisional Application No. 60/484,132, filed on Jul. 2, 2003. Priority to the aforementioned applications is hereby expressly claimed in accordance with 35 U.S.C. §§ 119, 120 and any other applicable statutes and the contents of each of the aforementioned applications are hereby incorporated herein by reference in their entirety.

### FIELD OF THE INVENTION

The field of the invention generally relates to crystalline forms of epirubicin hydrochloride, a compound which is useful as an anticancer chemotherapeutic drug. In particular, the field of the invention relates to a particular crystalline form of epirubicin hydrochloride which is distinguished by its improved thermal stability. In addition, the invention relates to methods of manufacturing the aforementioned crystalline form of epirubicin hydrochloride as well as to methods of using the aforementioned crystalline form of epirubicin hydrochloride to treat human and/or animal cancers.

### BACKGROUND OF THE INVENTION

Anthracyclines form one of the largest families of naturally occurring bioactive compounds. Several members of this family have shown to be clinically effective anti-neoplastic agents. These include, for example, daunorubicin, doxorubicin, idarubicin, epirubicin, pirarubicin, zorubicin, aclarubicin, and caminomycin. For instance, these compounds have shown to be useful in bone marrow transplants, stem cell transplantation, treatment of breast carcinoma, acute lymphocytic and non-lymphocytic leukemia, chronic lymphocytic leukemia, non-Hodgkin's lymphoma, and other solid cancerous tumors. U.S. Pat. Nos. 4,112,076, 4,345,068, 4,861,870, 5,945,518, and 5,874,550 disclose the preparation of epirubicin hydrochloride and its usage as an anticancer agent, which is represented by the formula:

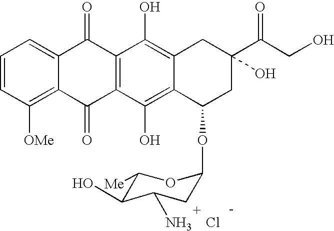

Active pharmaceutical ingredients (APIs) utilized for manufacturing of human medicines must be represented by pure and stable products. Requirements for purity and stability of the APIs are extremely important for pharmaceutical industry. One of the foremost methods of achieving product purity is to produce it in a specific crystalline form. A specific crystallization process is achieved by a combination of such parameters as solvent or a combination of solvents, pH, volume, temperature, and reaction duration. Variation of these parameters results in alternate crystalline forms of the same substance, so called polymorphs.

Currently, there are two major methods of recovery of epirubicin hydrochloride from solutions. The first method involves the treatment of the organic solution of epirubicin base with a solution of hydrogen chloride in methanol. See e.g., U.S. Pat. No. 4,112,076. Alternatively, the second method involves the precipitation of epirubicin hydrochloride from an aqueous or organo-aqueous solution with the aid of acetone. See e.g., U.S. Pat. No. 4,861,870.

U.S. Pat. No. 6,087,340 (“the '340 patent”) discloses an injectable ready-to-use solution containing epirubicin hydrochloride. More specifically, the '340 patent discloses a stable, injectable, sterile, pyrogen-free, anthracycline glycoside solution which consists essentially of a physiologically acceptable salt of an anthracycline glycoside dissolved in a physiologically acceptable solvent, which has a pH of from 2.5 to 3.5 and which is preferably contained in a sealed glass container. While the '340 patent discloses injectable, ready-to-use preparations, the No. 6,087,340 patent does not disclose the stabilization of epirubicin hydrochloride itself as a bulk drug.

U.S. Pat. No. 6,376,469 discloses a β-Type form of crystalline antharcycline amrubicin hydrochloride, which is produced by way of precipitation from acetone, acetonitrile and isopropanol, and which is said to have improved thermal stability. However, some time later, U.S. Pat. No. 7,091,469 discloses that the stability of such amrubicin is more likely related to formation of solvates than to the crystalline structure of the substance. U.S. Pat. No. 7,091,469 discloses data demonstrating the instability of the β-Type crystalline amrubicin and the formation of desaccharification and deamination products during its drying.

It is desirable, therefore, to have a crystalline form of epirubicin hydrochloride which has improved thermal stability characteristics. Variation of thermal stability for different crystalline forms of epirubicin hydrochloride is described for the first time herein.

### SUMMARY OF THE INVENTION

The present invention relates to a novel, strictly defined, crystalline form of epirubicin hydrochloride, named herein as “Type II” crystalline epirubicin hydrochloride, which has excellent thermal stability. Variation of thermal stability for different crystalline forms of epirubicin hydrochloride is described herein.

The inventors of the subject matter of the present invention previously disclosed a thermally stable “Type II” crystalline epirubicin hydrochloride and method of making the same in related U.S. patent application Ser. No. 10/877,221, and U.S. Provisional Application No. 60/484,132. Subsequent to those applications, the inventors have developed new Type II crystalline epirubicin hydrochloride and methods of making such new Type II epirubicin hydrochloride, as described in more detail herein.

Type II crystalline epirubicin hydrochloride is characterized by having a powder X-ray diffraction pattern having average values of diffraction angle (2θ) and relative intensity P(%) as presented in the table of FIG. 1. FIG. 1 represents a more detailed, X-ray diffraction spectrum than the one previously described in related U.S. patent application Ser. No. 10/877,221. However, all main diffraction maxima and their relative intensity are in the same proportion as was previously disclosed.

Accordingly, the present invention accomplishes several objectives, including:

(1) To provide a crystalline form (as well as method of making the same) of epirubicin hydrochloride, which is distinguished by improved thermal stability characteristics.

(2) To demonstrate dependence of the product purity of epirubicin hydrochloride from the method of crystallization.

(3) To provide an extraction method in which epirubicin hydrochloride is crystallized from the aqueous portion of an organo-aqueous solution.

(4) To provide an extraction method in which crystallization is conducted within the pH range of 2 to 5.

(5) To provide an extraction method in which crystallization is conducted at temperatures of 50-90° C.

(5) To provide an extraction method in which crystallization is conducted with hydrophilic organic solvents such as alcohols with carbon chains of C₁-C₃.

It is therefore an object of the invention to provide a crystalline form (i.e., type II) of epirubicin hydrochloride, which is distinguished from other crystalline forms of epirubicin hydrochloride by improved thermal stability characteristics and significantly higher purity, for example, see Table 7, and FIGS. 24 and 25). It is a further object of the invention to provide a method of synthesis for the aforementioned type II crystalline form of epirubicin hydrochloride.

### BRIEF DESCRIPTION OF THE DRAWINGS

FIG. 1 is a table of average values of diffraction angle (2θ) and relative intensity P(%) of type II crystalline epirubicin hydrochloride.

FIGS. 2-4 include Tables 2-4, respectively, illustrating the type II crystalline epirubicin hydrochloride XRD Analysis-Diffraction Angle (2θ) versus relative intensity (P %) for 3 separate commercial batches.

FIG. 5 include Table 5 illustrating a type I crystalline epirubicin hydrochloride XRD Analysis-Diffraction Angle (2θ) versus relative intensity (P %).

FIG. 6 includes Table 6 which shows chromatographic properties.

FIG. 7 includes Table 7 which shows the results of an assay comparison of epirubicin hydrochloride type I and epirubicin hydrochloride type II, and Table 8 which shows a comparison of their respective properties.

FIG. 8 includes Table 9 which shows the results of a thermal stability study of type II crystalline epirubicin hydrochloride.

FIG. 9 includes Table 10 which shows the results of a thermal stability study of type I crystalline epirubicin hydrochloride.

FIG. 10 illustrates a graph of the temperature vs. heat flow for type II crystalline epirubicin hydrochloride, according to one embodiment of the present invention.

FIG. 11 illustrates a graph of the temperature vs. heat flow for type I crystalline epirubicin hydrochloride, according to one embodiment of the present invention.

FIG. 12 illustrates an IR-spectrum plot of type II crystalline epirubicin hydrochloride, according to one embodiment of the present invention.

FIG. 13 illustrates an IR-spectrum plot of type I crystalline epirubicin hydrochloride.

FIG. 14 illustrates the powder x-ray diffraction spectrum of type I crystalline epirubicin hydrochloride (ESP04).

FIG. 15 and FIG. 16 illustrate the powder x-ray diffraction spectrum of type II crystalline epirubicin hydrochloride (Batch 181104) stored at room temperature and at accelerated storage conditions (40° C. for 3 months), respectively.

FIG. 17 and FIG. 18 illustrate the powder x-ray diffraction spectrum of type II crystalline epirubicin hydrochloride (Batch 191104) stored at room temperature and at accelerated storage conditions (40° C. for 3 months), respectively.

FIG. 19 and FIG. 20 illustrate the powder x-ray diffraction spectrum of type II crystalline epirubicin hydrochloride (Batch 201104) stored at room temperature and at accelerated storage conditions (40° C. for 3 months), respectively.

FIG. 21 illustrates an HPLC plot of the Test Solution of type I crystalline Epirubicin Hydrochloride injected immediately after preparation.

FIG. 22 illustrates an HPLC plot of the Test Solution of type I crystalline Epirubicin Hydrochloride injected 70 minutes after preparation.

FIG. 23 illustrates an HPLC plot of the Test Solution of type I crystalline Epirubicin Hydrochloride injected 140 minutes after preparation.

FIG. 24 illustrates an HPLC plot of the Test Solution of type II crystalline Epirubicin Hydrochloride injected immediately after preparation.

FIG. 25 illustrates an HPLC plot of the Test Solution of type II crystalline Epirubicin Hydrochloride injected 140 minutes after preparation.

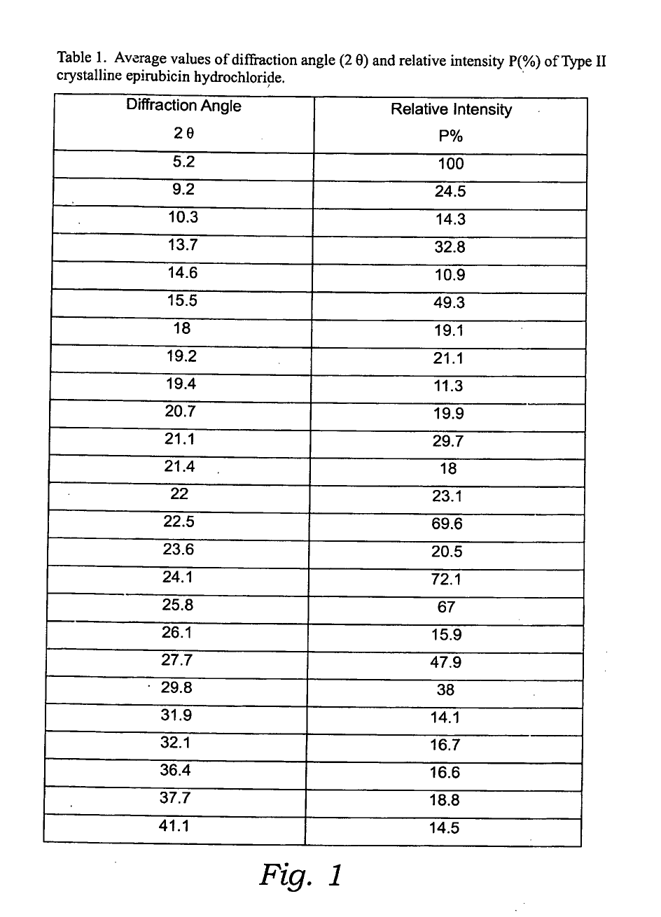

### DETAILED DESCRIPTION OF THE INVENTION

The present invention is directed to type II crystalline epirubicin hydrochloride which can be produced by crystallizing epirubicin hydrochloride from a suitable solvent such as, for example, water or mixture of water and a hydrophilic organic solvent. Preferably, crystallization of type II epirubicin hydrochloride is performed at a temperature of 50-90° C.

Crystallization is conducted by adding an alcohol with carbon chain C₁-C₃ to a solution of epirubicin hydrochloride in water or solvent-aqueous mixture. The pH of the solution is preferably maintained between 2 and 5.

Volume of the solvent preferably exceeds the volume of the epirubicin hydrochloride solution from 2 to 20 times. The crystallization process is conducted at temperatures from 50° C. to 90° C., preferably from 50° C. to 70° C.

Type II crystalline epirubicin hydrochloride obtained by this method is extracted by standard procedures known to those of ordinary skill in the art (e.g., vacuum-filtration through the filter or centrifugal filtration) followed by drying of the crystals. The produced type II crystalline epirubicin hydrochloride can be used for preparation of the final dosage forms of epirubicin hydrochloride. The following two examples disclose methods of producing type II crystalline epirubicin hydrochloride.

### EXAMPLE 1

(1) A solution of epirubicin hydrochloride (10.0 grams) in water or in ethanol-in-water mixture (pH 3-4) undergoes low-pressure evaporation at a temperature of 40° C. until a gel state of the solution is achieved.

(2) 1-propanol in the amount of 12-times the volume of the residual solution is then added to the residual solution and stirred for 3 hours at a temperature of 60° C.

(3) Precipitated crystals of epirubicin hydrochloride are then collected by filtration, washed in 10 ml of acetone and dried at room temperature.

(4) The result is 9.3 grams of type II epirubicin hydrochloride crystals.

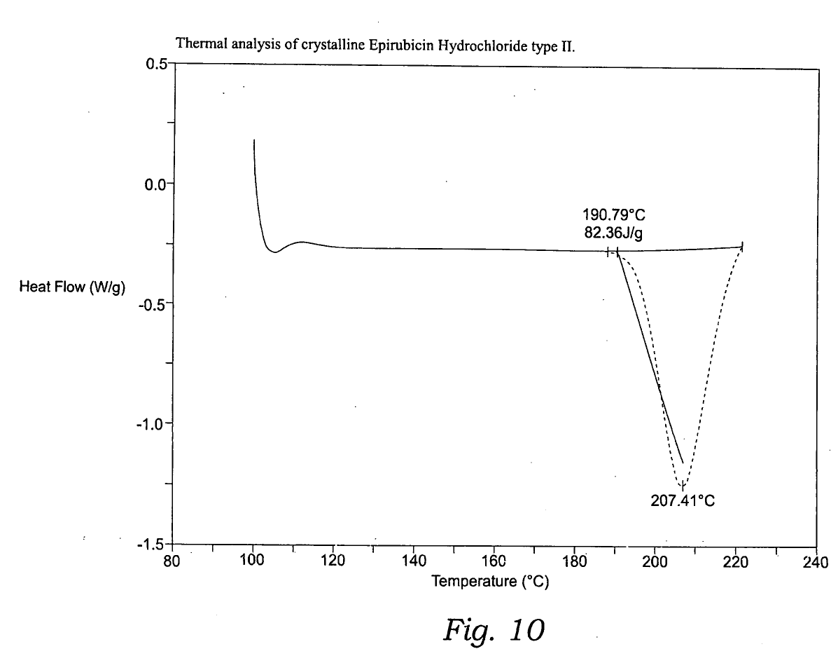

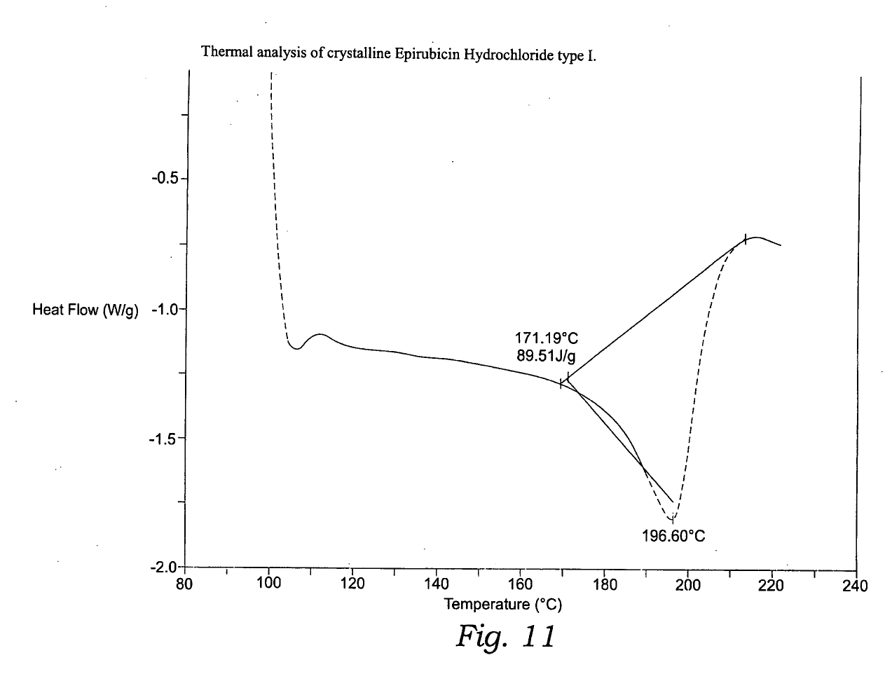

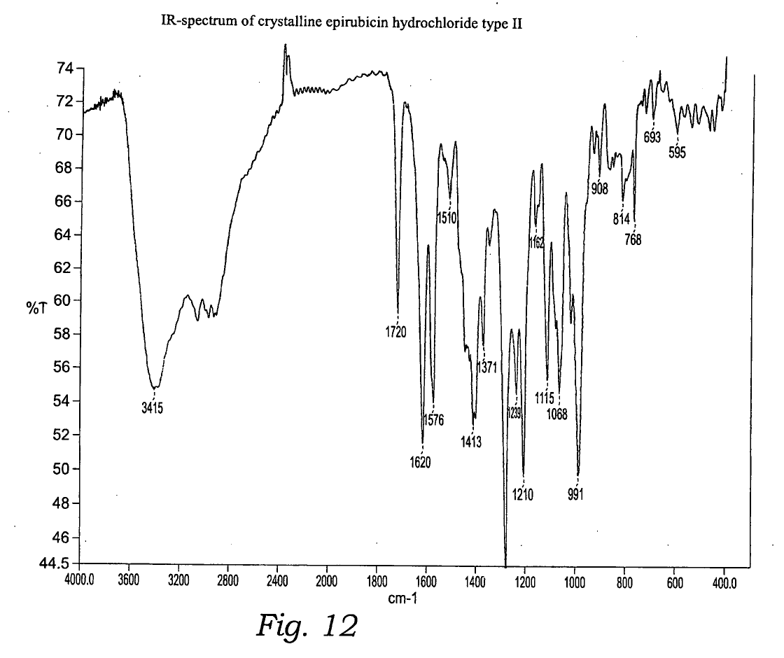

(5) As seen in FIG. 10, the melting point of type II crystalline epirubicin hydrochloride is approximately 207° C. with decomposition (hot stage 2° C./min). FIG. 12 illustrates the IR-spectrum (IR (KBr)) of type II crystalline epirubicin hydrochloride. Peaks/valleys are seen at 3415, 2928, 1720, 1620, 1576, 1510, 1413, 1371, 1284, 1239, 1210, 1162, 1115, 1068, 1019, 991, 930, 908, 880, 814, 768, 719, 693, and 595 cm⁻¹.

### EXAMPLE 2

(1) A solution of epirubicin hydrochloride (10.0 grams) in water or in ethanol-in-water mixture (pH 3-4) undergoes low-pressure evaporation at a temperature of 40° C. until a gel state of the solution is achieved.

(2) Absolute ethanol in the amount of 10-times the volume of the original solution is then added to the residual solution and stirred for 2 hours at a temperature of 60° C.

(3) Precipitated crystals of epirubicin hydrochloride are then collected by filtration, washed in 10 ml of ethanol and 10 ml of acetone and dried at room temperature.

(4) The result is 7.5 grams of type II epirubicin hydrochloride crystals.

The following example (Example 3) discloses a method of producing type I epirubicin hydrochloride crystals, namely epirubicin hydrochloride crystals as described in U.S. Pat. No. 4,861,870.

### EXAMPLE 3

(1) Step is identical to step 1 in Example 1 above.

(2) Gel solution of epirubicin hydrochloride is poured into 300 ml of acetone.

(3) Precipitated crystals of epirubicin hydrochloride are then collected by filtration and washed in 50 ml of acetone.

(4) The result is 9.7 grams of type I epirubicin hydrochloride crystals.

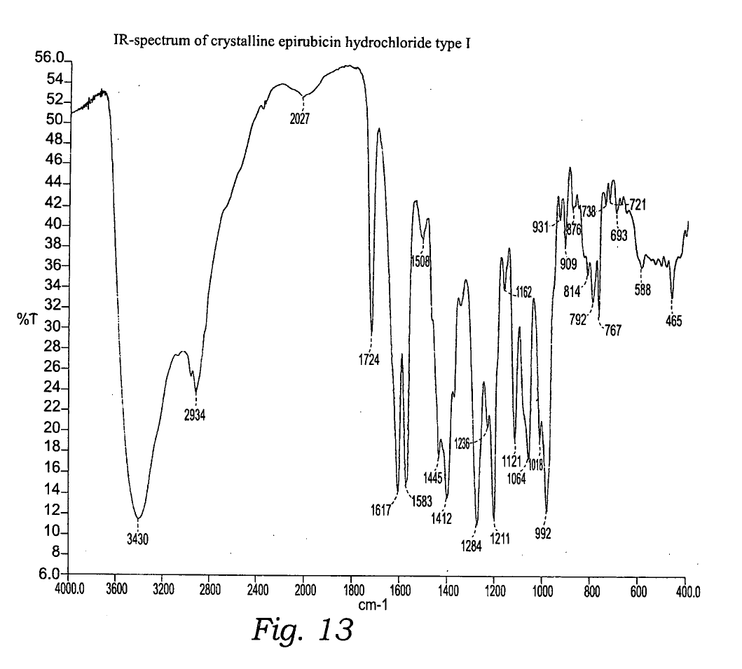

As seen in FIG. 12, the melting point of type I crystalline epirubicin hydrochloride is approximately 196° C. with decomposition (hot stage 2° C./min). FIG. 13 illustrates the IR-spectrum (IR (KBr)) of type I crystalline epirubicin hydrochloride. Peaks/valleys are seen at 3430, 2934, 2027, 1724, 1617, 1583, 1508, 1445, 1412, 1284, 1236, 1211, 1162, 1121, 1064, 1018, 992, 931, 909, 876, 814, 792, 767, 738, 721, 693, 588, and 465 cm⁻¹.

### EXAMPLE 4

Optical Microscopy was performed on type I and II crystalline epirubicin hydrochloride as described below:

Microscope used: Labomed CXRIII optical microscope with polarizing filters. The samples of epirubicin hydrochloride obtained in Example 1 (type II) and Reference Example 3 (type I) both exhibit birefringence and are, therefore, anisotropic crystals.

### EXAMPLE 5

In this example, powder X-ray diffraction spectra of crystalline epirubicin hydrochloride of type I and type II were obtained. Powder X-ray diffraction spectra were measured using a Rigaku Cu Anode X-ray Diffractometer (MiniFlex). The conditions for analysis of the samples was as follows:

Start angle: 3

Stop angle: 90

Sampling: 0.02

Scan speed: 1.00

X-ray powder diffraction performed with Copper Kα (γ=1.5406 Å incident X-ray)

Vertical θ: 2θ Bertrano Parafocusing Diffractometer

Nil scintillating (Pulse height PMT) detector

Kβ Nickel filter

The results of the measured powder X-ray diffraction spectra are as follows:

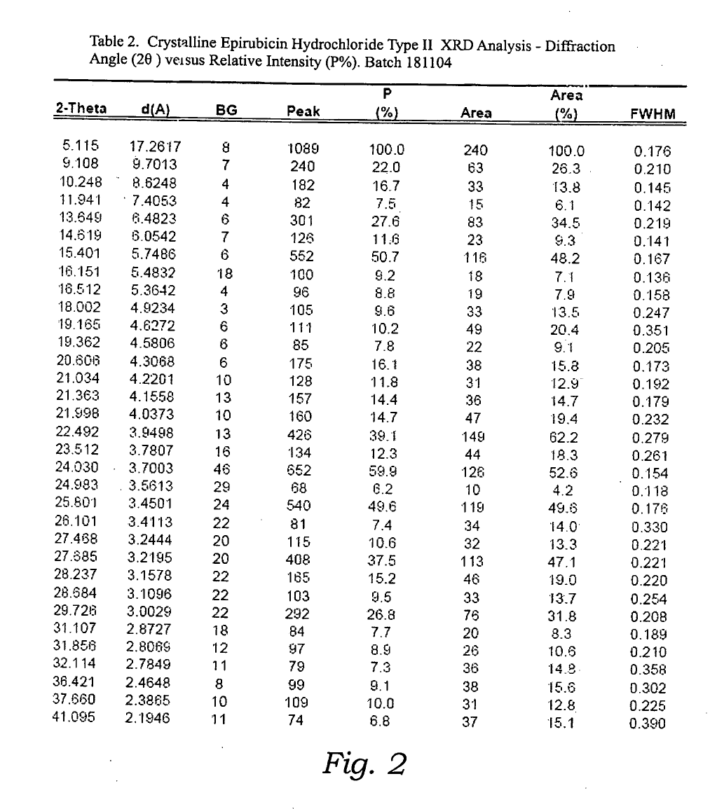

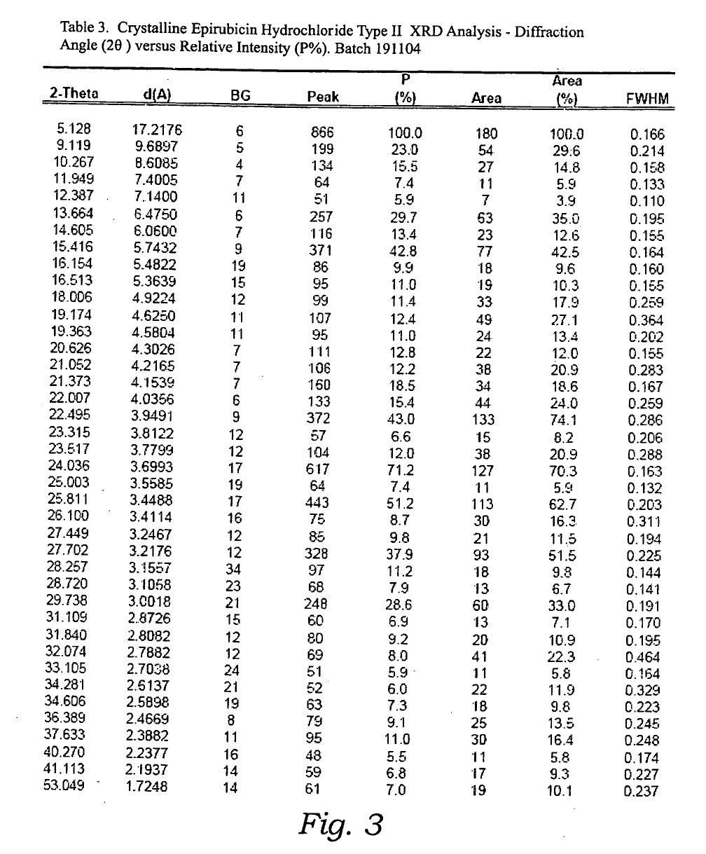

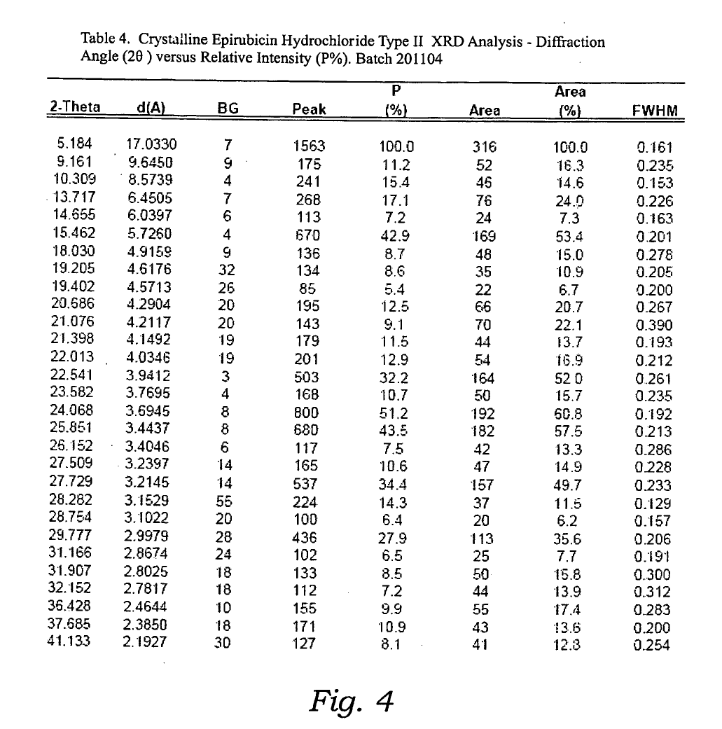

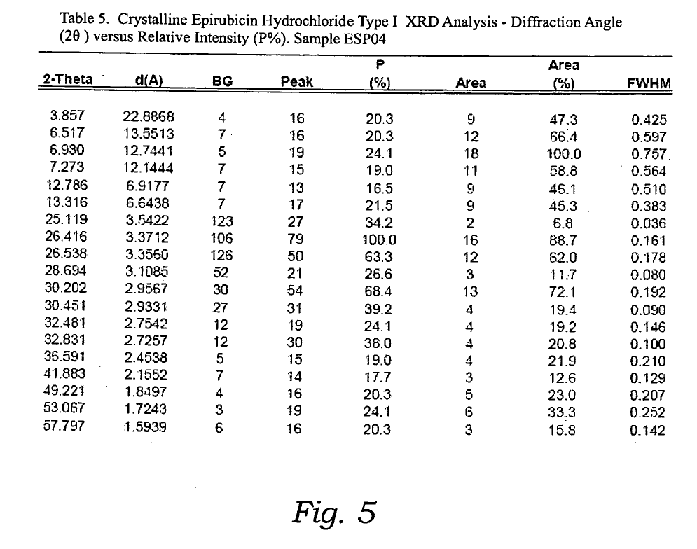

The X-ray diffraction patterns are dissimilar for the samples obtained in Example 1 (Type II) and Reference Example 3 (Type I). FIGS. 2-4 include Tables 2-4 which illustrate the type II crystalline epirubicin hydrochloride XRD Analysis-Diffraction Angle (2-Θ) versus Relative Intensity (P %) for 3 commercial batches (181104, 191104, 201104). In contrast, Table 5 shown in FIG. 5 illustrates the type I crystalline epirubicin hydrochloride XRD Analysis-Diffraction Angle (2-Θ) versus Relative Intensity (P %) for sample ESP04, based on methods described in Example 3 (Reference).

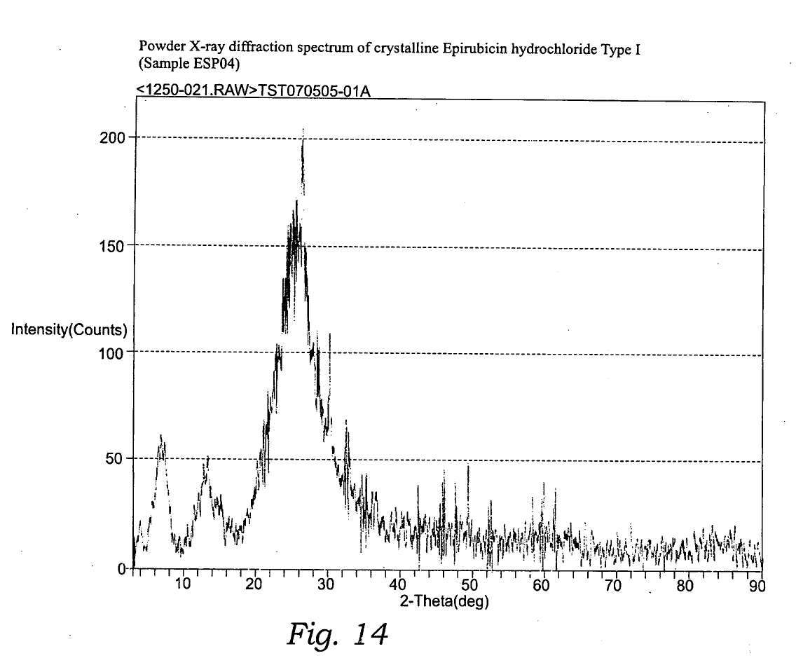

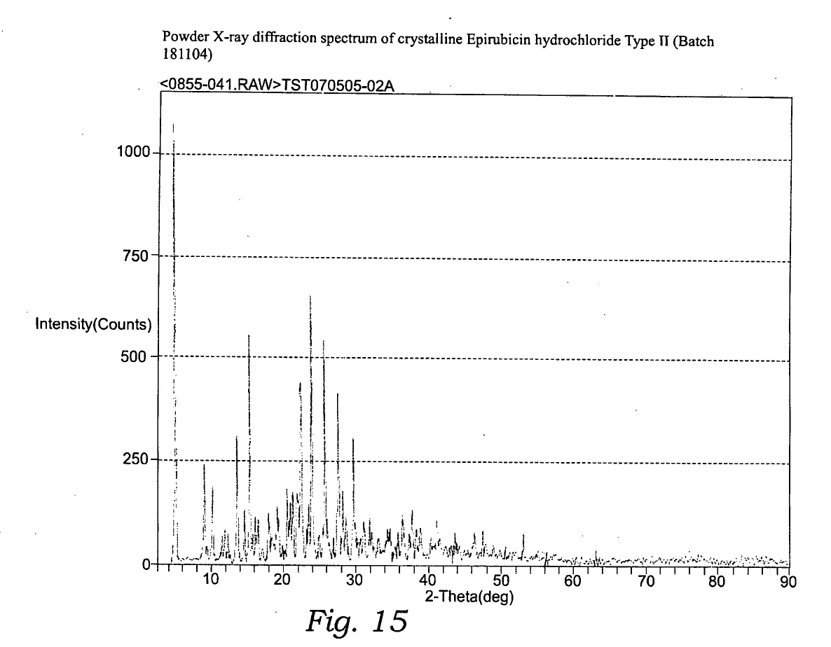

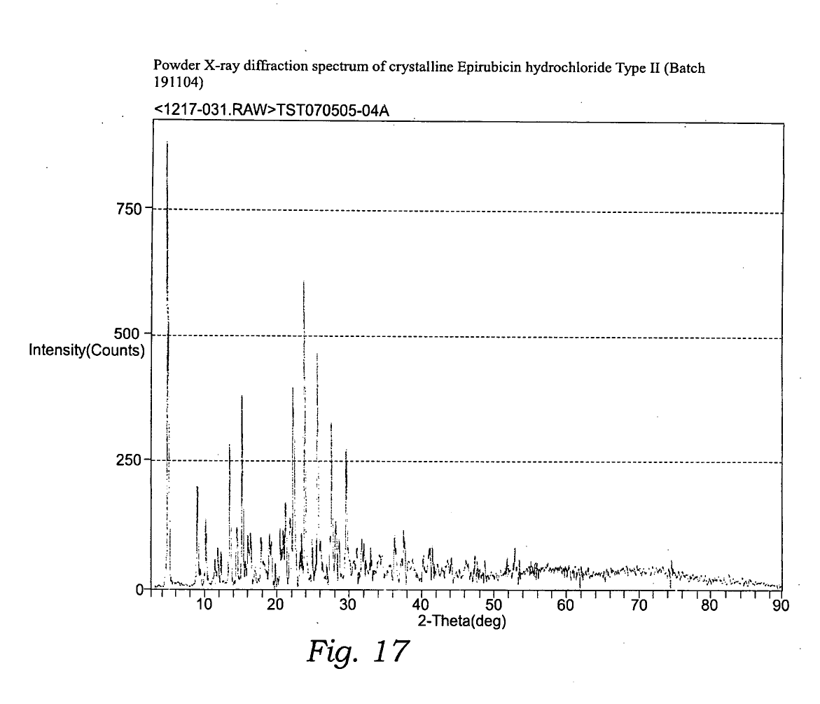

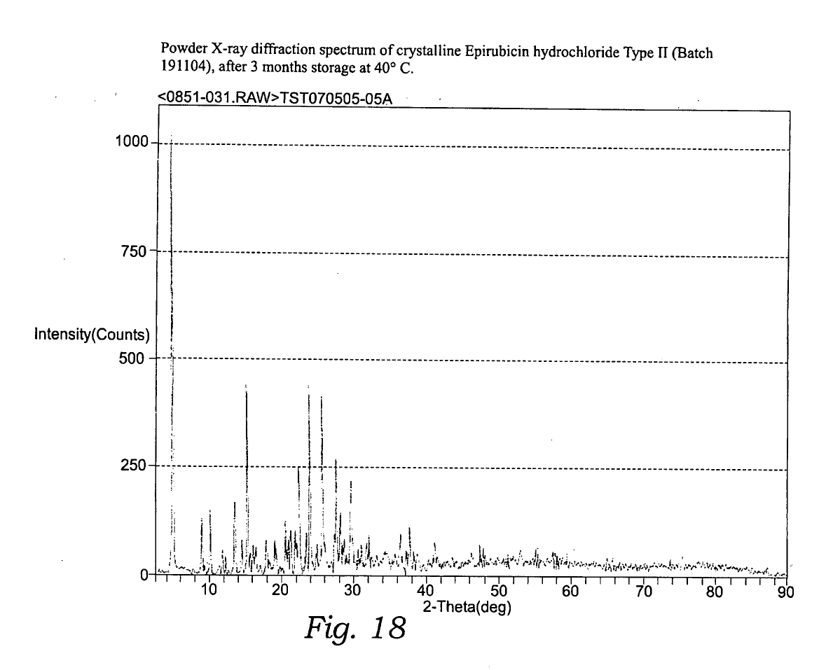

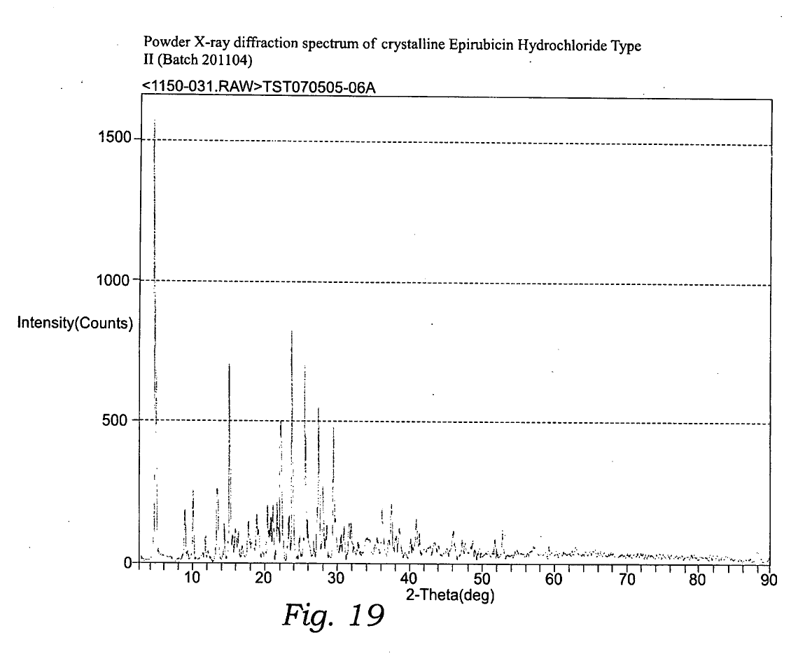

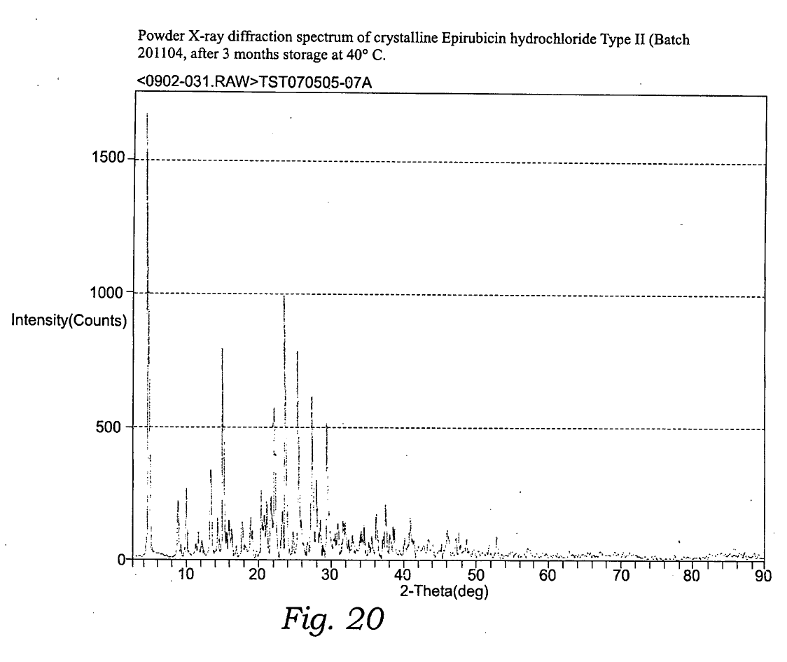

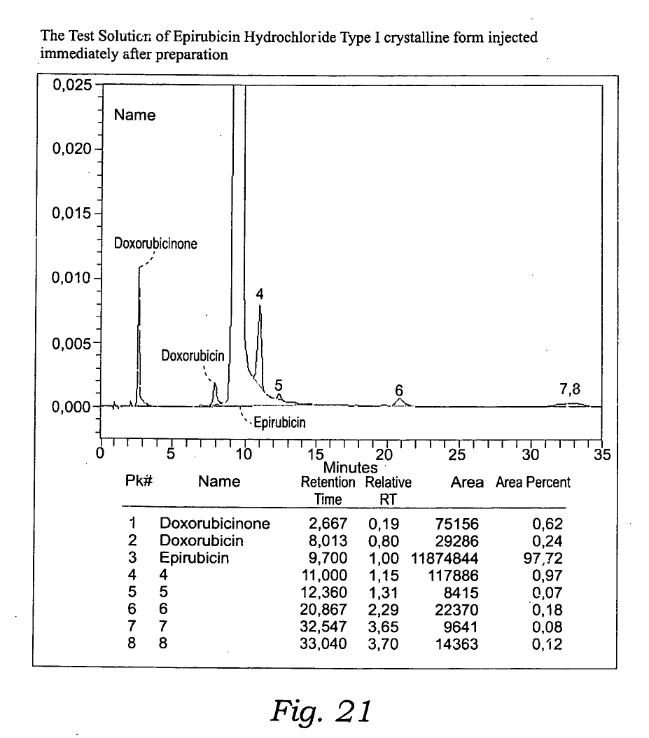

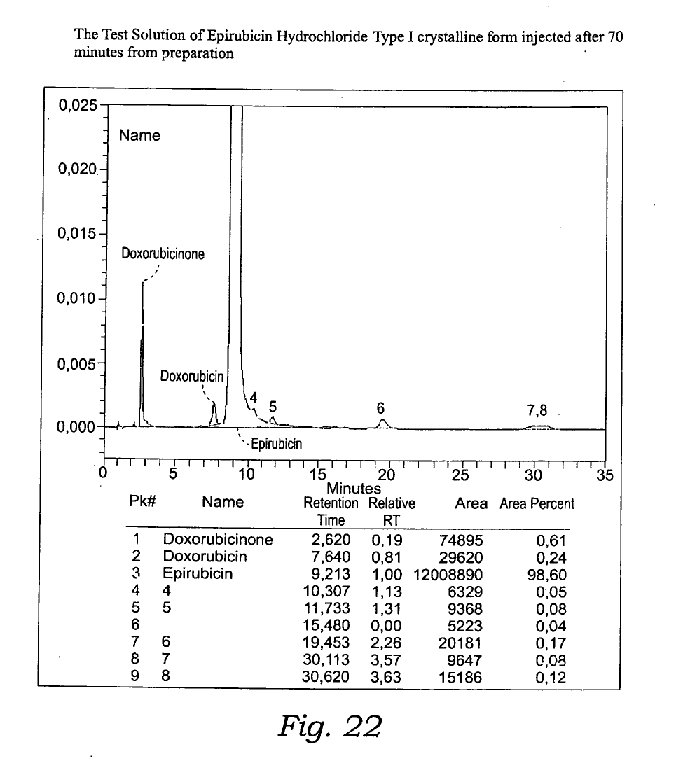

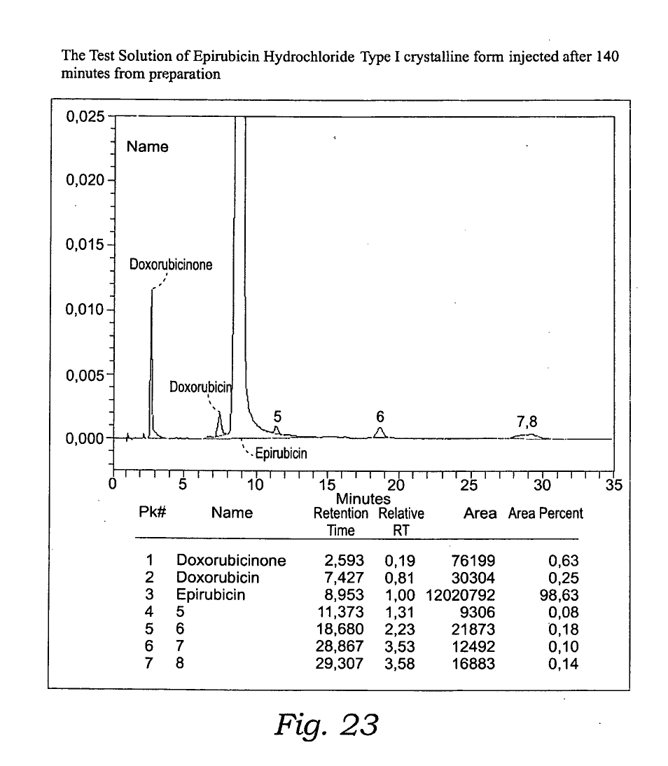

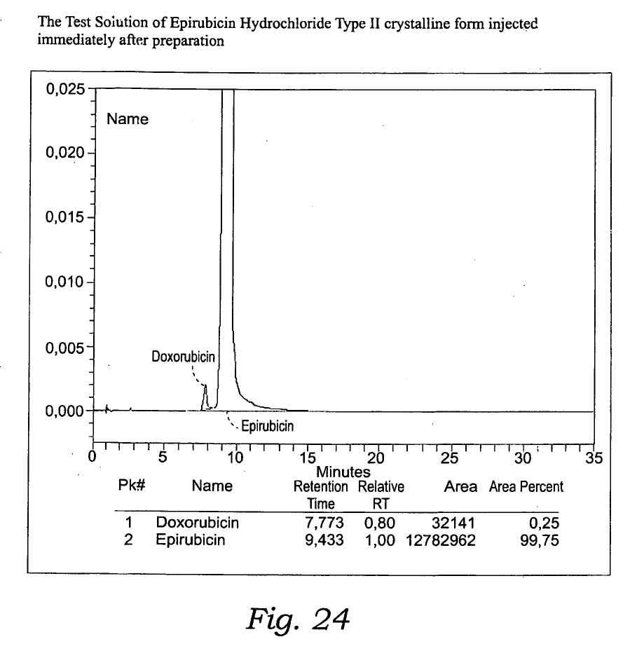

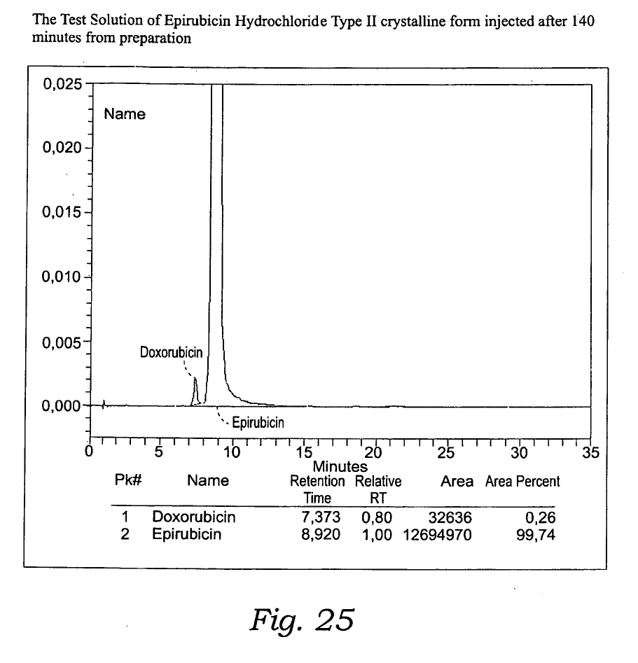

Type I crystalline epirubicin hydrochloride gives a single strong signal at approximately 24.6 degrees. In contrast, type II crystalline epirubicin hydrochloride gives multiple strong signals across the entire spectrum. FIGS. 15-20 illustrate the powder X-ray diffraction spectra of type II crystalline epirubicin from 3 consecutive commercial batches of epirubicin, based on the methods described in Example 1 (Reference). FIG. 14 illustrates the powder X-ray diffraction spectrum of type I crystalline epirubicin hydrochloride obtained in Example 3 (Reference).

### EXAMPLE 6

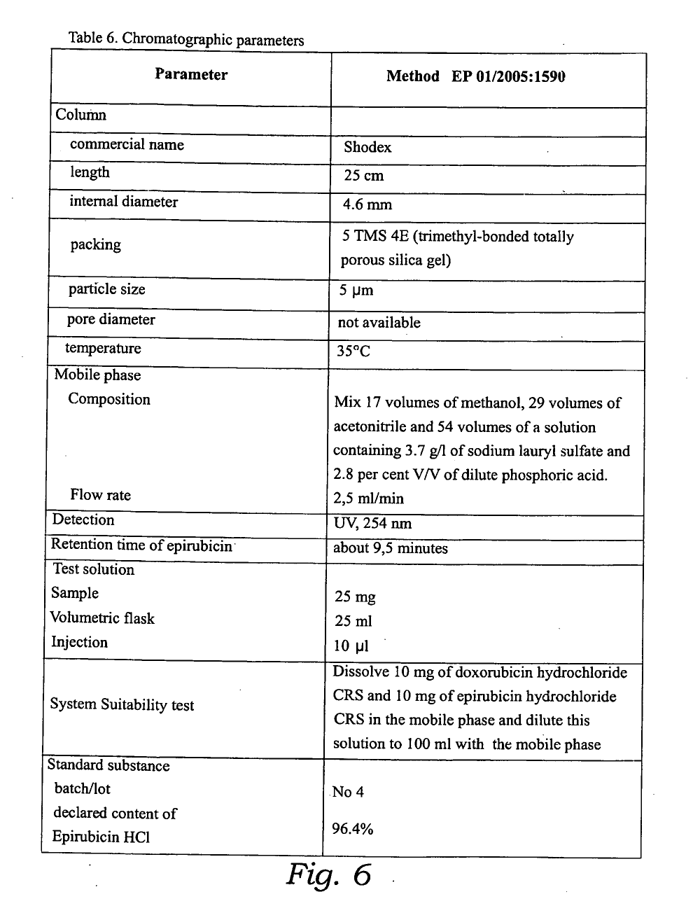

The properties of Epirubicin Hydrochloride Type I crystalline form (methods described in Example 3) and Epirubicin Hydrochloride Type II crystalline form (methods described in Example 1) were investigated and compared against EDQM Chemical Reference Substance (CRS) Epirubicin Hydrochloride. The FP HPLC method was used for this investigation. The parameters of this method are given in Table 6 shown in FIG. 6.

The following Results were obtained:

A. Related Substances

On the chromatograms of Epirubicin Hydrochloride Type II crystalline form, only a single peak is detected (0.25-0.30%) which is due to a related substance (Doxorubicin). There are seven impurity peaks on the chromatograms of Epirubicin Hydrochloride Type I crystalline form; total area % of them varies from 1.35% to 2.34%.

Another characteristic of Epirubicin Hydrochloride Type I crystalline form is the instability of the peak #4 in EP system, RRT 1.15 (Pic 1, 2). On the chromatograms of the first injections of each Epirubicin Hydrochloride Type I solution, its area % is 0.55 to 0.97. At the second injection, its area % is almost negligible, and at the third injection it disappears. The area % of each of the other impurity peaks remains practically unchanged, but the area of the epirubicin peak at the second injection is always larger than at the first injection. No peak with such behaviour is detected in Epirubicin Hydrochloride Type II. We can conclude that this peak is due to an unstable epirubicin complex with acetone which decomposes in a solution and is converted into epirubicin.

B. Assay

Epirubicin Hydrochloride Type I and Epirubicin Hydrochloride Type II were quantitatively compared. For both samples the assay was calculated against EDQM CRS.

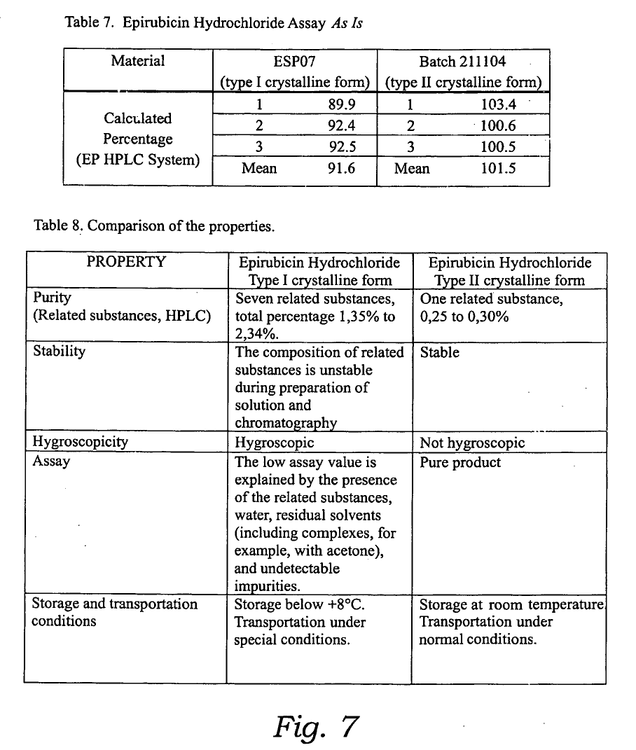

The results are given in Table 7 shown in FIG. 7.

Thus, the quantitative content of epirubicin hydrochloride in the crystalline epirubicin Type II is almost 10% higher than that of the epirubicin Type I. Since the quantity of the related substances varies from 1.35% to 2.34%, the remaining difference is comprised of undetected impurities.

Table 8 shown in FIG. 7 compares and contrasts the characteristics of epirubicin type I and epirubicin type II.

### EXAMPLE 7

The following example illustrates the improved thermal stability of type II crystalline epirubicin hydrochloride as compared to type I crystalline epirubicin hydrochloride.

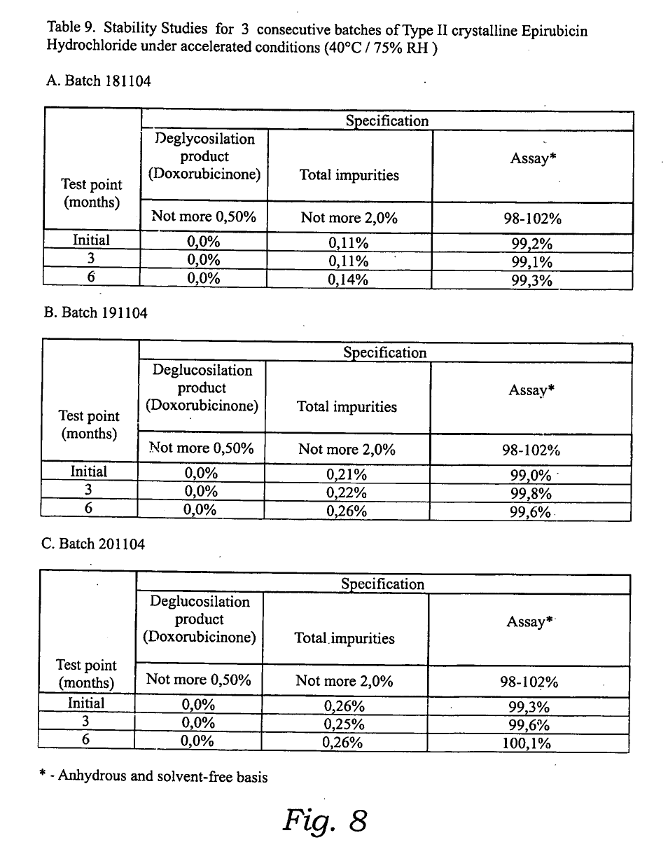

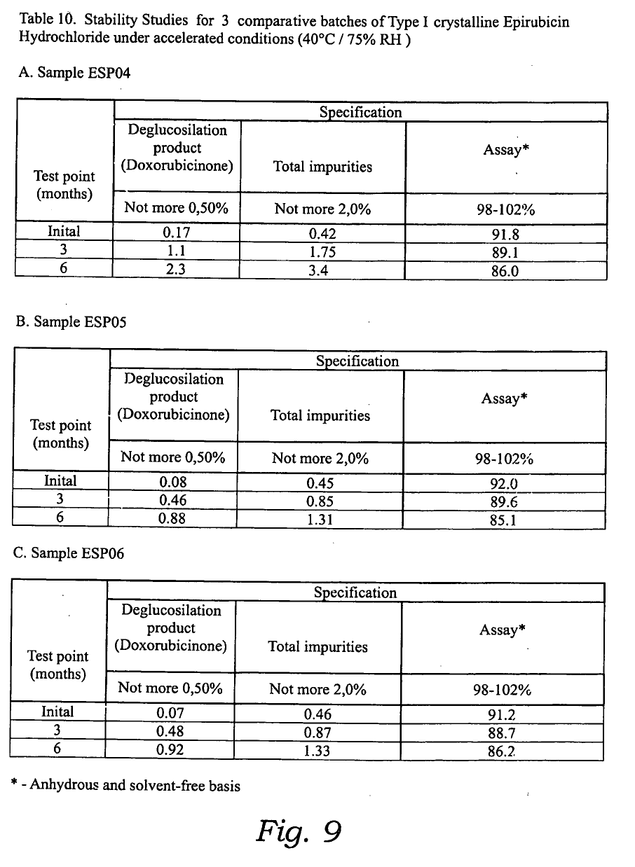

The type II crystalline epirubicin hydrochloride obtained in Example 1 and type I crystalline epirubicin hydrochloride obtained in reference Example 3 were each kept at a temperature 40° C. for six months, thereby simulating accelerated storage conditions. The thermal stability was investigated and measured by studying the following parameters: (1) assay (HPLC method), (2) doxorubicinone quantity (doxorubicinone, an aglycone of epirubicin, is the major epirubicin degradation product), and (3) total impurities. The results of this investigation are presented in Table 9 shown in FIG. 8 and Table 10 shown in FIG. 9.

As the results in Tables 9 and 10 confirm, type II crystalline epirubicin hydrochloride exhibits much greater thermal stability than type I crystalline epirubicin hydrochloride. This is particularly advantageous because the type II crystalline epirubicin hydrochloride will retain its efficacy for a longer period of time as compared to type I crystalline epirubicin hydrochloride because there is less degradation and impurities. This also means that the shelf life of type II crystalline epirubicin hydrochloride is longer than the shelf life of type I crystalline epirubicin hydrochloride.

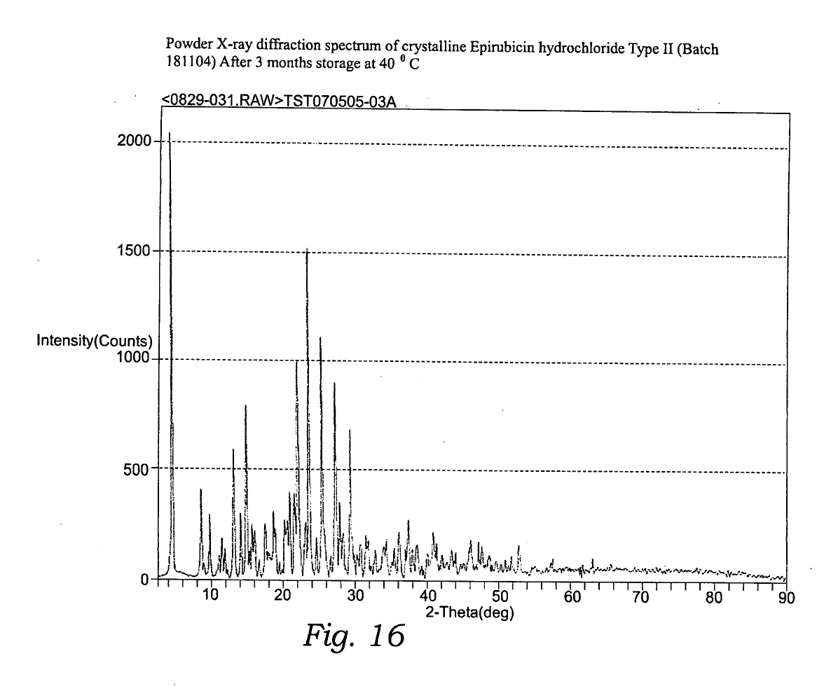

FIGS. 16, 18 and 20 illustrate the X-Ray Diffraction spectra of the mentioned above 3 commercial batches, undergone 6 months of Accelerated storage conditions (40° C.). This data unequivocally confirms that type II crystalline epirubicin is thermally stable.

While embodiments of the present invention have been shown and described, various modifications may be made without departing from the scope of the present invention. The invention, therefore, should not be limited, except to the following claims, and their equivalents.

We claim:

1. A crystalline epirubicin hydrochloride, having a powder X-ray diffraction pattern having average values of diffraction angle (2θ) and relative intensity (P(%)) substantially as presented in the following table:

| Diffraction Angle | Relative Intensity |
|---|---|
| 2Θ | P % |
| 5.2 | 100 |
| 9.2 | 24.5 |
| 10.3 | 14.3 |
| 13.7 | 32.8 |
| 14.6 | 10.9 |
| 15.5 | 49.3 |
| 18 | 19.1 |
| 19.2 | 21.1 |
| 19.4 | 11.3 |
| 20.7 | 19.9 |
| 21.1 | 29.7 |
| 21.4 | 18 |
| 22 | 23.1 |
| 22.5 | 69.6 |
| 23.6 | 20.5 |
| 24.1 | 72.1 |
| 25.8 | 67 |
| 26.1 | 15.9 |
| 27.7 | 47.9 |
| 29.8 | 38 |
| 31.9 | 14.1 |
| 32.1 | 16.7 |
| 36.4 | 16.6 |
| 37.7 | 18.8 |
| 41.1 | 14.5 |

2. A process of preparing a crystalline epirubiucin hydrochloride according to claim 1 comprising crystallization of epirubcicin hydrochloride at a temperature of between 50° C.-90° C.

3. A process according to claim 2 comprising crystallization of epirubicin hydrochloride from a hydrophilic organic solvent comprising an alcohol with carbon chain C₁-C₃, and a solution of epirubicin hydrochloride in one of the following: water, or a mixture of hydrophilic organic solvent in water.

4. A process of preparing a crystalline epirubiucin hydrochloride according to claim 1 comprising:
a. dissolving epirubicin hydrochloride in water or in a mixture of hydrophilic organic solvent in water to form a solution;
b. adjusting the pH of the solution to a value between 3 and 4.
c. evaporating the solution at a temperature of about 40° C. until the solution is in a gel state; and
d. crystallizing epirubicin hydrochloride by adding a hydrophilic organic solvent at a temperature of between 50° C.-90° C.
